(19) United States
(12) Patent Application Publication
(10) Pub. No.: US 2020/0215149 A1
(43) Pub. Date: Jul. 9, 2020

(54) N-Phenylacetyl-L-prolylglycine ethyl ester compositions and methods for using the same

(71) Applicant: Christopher David Thompson, Santa Monica, CA (US)

(72) Inventor: Christopher David Thompson, Santa Monica, CA (US)

(21) Appl. No.: 16/242,982

(22) Filed: Jan. 8, 2019

(51) Int. Cl. A61K 38/05; A61K 31/047; A61K 9/00; A61K 9/68; A61K 31/7068; A61K 31/522

(52) U.S. Cl. CPC A61K 38/05; A61K 31/047; A61K 9/0053; A61K 31/522; A61K 9/0056; A61K 31/7068; A61K 9/0058

(57) ABSTRACT

A composition comprising a combination of N-Phenylacetyl-L-prolylglycine ethyl ester and cannabidiol for oral administration to humans and other animals and a method for administering said composition are described. The composition provides synergistic nootropic benefits to the user, resulting in enhanced cognitive functions, such as increased focus, creativity, memory recall, and memory consolidation, among other cognitive functions. The composition generally does not cause side effects in the users and generally does not cause the user to develop tolerance to its nootropic effects. The composition optionally comprises a compound containing choline or a choline prodrug to supply choline to the brain of the user for the production of the neurotransmitter acetylcholine, and may also optionally comprise caffeine.

### CROSS-REFERENCE TO RELATED APPLICATIONS

Not applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC

Not Applicable

### BACKGROUND OF THE INVENTION

The field of the invention is N-Phenylacetyl-L-prolylglycine ethyl ester compositions and methods for using the same.

“Nootropics” are a group of compounds and mixtures thereof whose consumption has been shown to enhance some cognitive functions, often by affecting neurotransmitters involved in cognitive processes. This enhancement of cognitive functions is attained by the consumption of a dose of the nootropic that exceeds a certain minimum threshold. This threshold dose is hereafter referred to as an “effective dose,” which may depend on the type of nootropic and on the user. In some cases, during a period of regular consumption of the nootropic, the user may develop a tolerance to the nootropic, such that, to attain a similar enhancement of cognitive functions, the user may need to consume an increasingly larger dose of the nootropic over the course of the period of regular consumption. In these cases, the effective dose of the nootropic for the user increases over time.

It is possible that the effective dose of a nootropic substance be so large as to cause the user to experience uncomfortable and unwanted side effects along with the nootropic effects. Possible side effects caused by nootropic substances and unwanted by users may comprise but are not limited to anxiety, irritability, disturbed sleep, and higher blood pressure. The large mass of the effective dose of such nootropics may also cause the user discomfort during administration due to the unpleasant taste of many nootropics. Additionally, nootropics with larger effective doses require more space to ship the same number of doses, as well as to store the same number of doses, resulting in a higher overall cost compared to nootropics with smaller effective doses. Therefore, a smaller effective dose is typically preferred by users and suppliers alike.

The synthesis of the nootropic compound N-Phenylacetyl-L-prolylglycine ethyl ester was first reported in 1996 (Gudasheva, et al. (1996) Eur. J. Med. Chem. 31:151-157). This compound will hereafter be referred to by its common name “noopept.” Noopept's lack of a 2-oxo-pyrollidine nucleus makes it chemically distinct from “racetams,” a common class of nootropic compounds. Noopept has been shown to have a smaller effective dose than some racetams, such as piracetam (Neznamov and Teleshova (2009) Neurosci. Behay. Physiol. 39:311-321).

Nootropic stimulants, such as methylphenidate and amphetamines, are commonly consumed by healthy adult humans whose work or responsibilities require high levels of cognitive function for enhancement of cognitive functions, such as college students and surgeons (Fond, et al. (2015) J. Clinic. Res. Bioeth. 6:1000213). Nevertheless, there are concerns about the side effects that such stimulants may cause in healthy adult humans. Some of these stimulants may cause harm to the central nervous system, heart, and muscles, although the effects vary among users (d'Angelo, et al. (2017) Br. J. Pharmacol. 174:3257-3267).

It would be desirable to provide a nootropic composition that is capable of enhancing cognitive functions without causing the user adverse side effects. It would also be desirable for the dose of said nootropic composition to be smaller for more comfortable consumption and reduced costs of shipping and storage.

### BRIEF SUMMARY OF THE INVENTION

The present invention is a composition comprising a combination of noopept and cannabidiol. The present invention is based, in part, on the discovery of the synergistic nootropic effect resulting from the consumption of the combination of noopept and cannabidiol, which enhances cognitive functions of the brains of humans and of other animals, generally without causing tolerance or adverse side effects.

In addition to enhancing mental cognition, the disclosed composition possesses several features that are preferred among users. One preferred feature of the disclosed composition is that the effective dose of the disclosed composition is generally lower than the effective dose of other nootropic compositions. As a non-limiting example, the effective dose of noopept may be over 50 times smaller in mass than the effective dose of piracetam (Neznamov and Teleshova (2009) Neurosci. Behay. Physiol. 39:311-321). A smaller effective dose may be preferable for some users due to the greater ease of consuming smaller quantities of a substance, as well as due to the lower price resulting from the lower cost of shipping and storing smaller quantities of a substance. Furthermore, studies on noopept as well as studies on cannabidiol have found that users generally do not develop a tolerance to the nootropic effects of either compound (Ostrovskaya, et al. (2008) Bull. Exp. Biol. Med. 146:334-337; Hayakawa, et al. (2007) Neuropharmacology 52:1079-1087).

In one embodiment, the disclosed composition also comprises a compound containing choline or a choline prodrug. The consumption of a compound containing choline or a choline prodrug results in higher levels of choline in the brain of the user. Choline potentiates the nootropic effect of noopept, such that an embodiment of the composition to which choline is added may enhance some cognitive functions more than such an embodiment to which choline is not added.

In an additional embodiment, the disclosed composition further comprises caffeine. Caffeine is a stimulant, which may enhance some cognitive functions, particularly mental attention, alertness, reaction time, and mental focus. Users that consume this particular embodiment of the disclosed composition, however, may develop a tolerance to the caffeine in the composition.

In a further embodiment, the disclosed composition is administered orally in admixture with a carrier or excipient comprising other components that may be inactive or active. These components are intended to be, but are not limited to being, non-toxic and not greatly inhibiting of the nootropic effects of the disclosed composition on the user.

In an additional embodiment, the present invention is also a dietary supplement comprising the disclosed composition for oral administration.

In yet another embodiment, the disclosed composition is administered according to a regimen, in which the daily dose at the beginning of the regimen is elevated relative to the daily dose later in the regimen, commonly referred to as a regimen comprising a “loading phase.”

### BRIEF DESCRIPTION OF THE SEVERAL VIEWS OF THE DRAWING

Not Applicable

### Definitions

The term “noopept” refers to N-Phenylacetyl-L-prolylglycine ethyl ester, a compound that has been shown to have nootropic benefits (Neznamov and Teleshova (2009) Neurosci. Behay. Physiol. 39:311-321).

The term “CBD” refers to cannabidiol, which may be extracted from hemp, cannabis, or other natural sources, as well as derived synthetically.

The term “high-concentration choline source” refers to a substance that, when introduced into the body of a user, can result in the concomitant introduction into the bloodstream of the user of an amount of choline with a mass totaling at least 10% of the mass of the substance. As a non-limiting example, the substance may be a prodrug for choline, such as citicoline, or may contain choline, such as L-Alpha glycerylphosphorylcholine (hereafter referred to as “alpha GPC”).

The term “ACh” refers to acetylcholine, a neurotransmitter.

The term “effective dose” refers to the minimum threshold for the dose of a nootropic substance, such that doses exceeding this threshold in quantity are sufficient to enhance the cognitive functions of the user. The effective dose of a nootropic substance may be specific to the substance as well as the user, and therefore may vary for different substances as well as for different users. The effective dose for a user may be determined on the basis of experimentation or through predictions based on empirical evidence. For a substance to which a user may develop tolerance, the effective dose may increase following regular consumption of the substance.

The term “nootropic substance” refers to a substance that is capable of enhancing “cognitive functions” when introduced into the body of a user.

The term “cognitive functions” refers to capabilities enabled by the brain of an individual. These functions comprise but are not limited to reasoning abilities, clarity of thought, verbal fluency, reaction time, speed of processing information, short-term memory access, memory consolidation, creativity, attention, alertness, mental stamina, and mental energy, as well as other capabilities enabled by the brain of the individual.

The term “user” refers to a human or other animal, at least one of whose cognitive functions may be enhanced upon receiving the disclosed composition through oral administration.

The term “loading phase” refers to a part of a regimen designed to guide the consumption of the disclosed composition, during which the daily consumption of the disclosed composition is elevated relative to the daily consumption of the disclosed composition during the part of the regimen that is not the “loading phase.”

The term “flow” refers to a state of mind in which creativity and mental focus are enhanced (Csikszentmihalyi (2014) Flow and the Foundations of Positive Psychology, pp. 209-226), often marked by an increase in the amplitude of alpha brain waves, which typically range from 7.5-12.5 Hz in frequency (Nah, et al. “A Review on Neurophysiological Correlates of Flow,” HCIBGO 2017, pp. 364-372).

### DETAILED DESCRIPTION OF THE INVENTION

In accordance with the present invention, a composition comprising noopept and CBD is orally administered to a user to enhance the cognitive functions of the user. In particular, the composition may promote a state of focus and flow in the mind of the user. The quantity of the noopept and the quantity of the CBD are generally, but not necessarily, equal to or in excess of the effective dose required for the enhancement of the cognitive functions of the user. In particular, when administered in combination with noopept, CBD potentiates the nootropic benefits provided by noopept, such that a composition comprising both CBD and noopept can enhance some cognitive functions to a greater extent than the same composition without CBD or the same composition without noopept.

In a preferred embodiment, the composition is particularly intended to enhance some cognitive functions in a user whose brain is healthy, although the composition may also enhance some cognitive functions in users with neurodegenerative diseases, mental illness, or neurological damage, among other afflictions and maladies that hinder cognitive function.

Some studies have provided evidence of the capability of the disclosed composition to enhance some cognitive functions by measuring alpha brain waves. Alpha brain waves typically range from 7.5-12.5 Hz in frequency and have been shown to be positively correlated with flow (Nah, et al. “A Review on Neurophysiological Correlates of Flow,” HCIBGO 2017, pp. 364-372). In these studies, subjects that consumed the disclosed composition experienced an increase in the amplitude of alpha brain waves as compared to subjects that consumed a placebo, showing that consumption of the disclosed composition can enhance the cognitive functions of the user.

The composition generally does not cause the user to experience adverse side effects or tolerance to the nootropic effects of the composition. As a non-limiting example, some studies have shown that noopept causes fewer side effects in healthy human subjects than piracetam, a common nootropic compound (Neznamov and Teleshova (2009) Neurosci. Behay. Physiol. 39:311-321). Some studies have also shown that CBD generally does not cause significant side effects in humans who consume effective doses of CBD (Bergamaschi, et al. (2011) Current Drug Safety 6:237-249). Furthermore, neither noopept nor CBD has been shown to cause the user to become tolerant or addicted to either compound (Ostrovskaya, et al. (2008) Bull. Exp. Biol. Med. 146:334-337; Hayakawa, et al. (2007) Neuropharmacology 52:1079-1087). It is thus generally preferable to a user to use the disclosed composition over using a different nootropic composition that results in a greater number or degree of adverse side effects, or that causes the user to develop tolerance to its nootropic effects.

The disclosed composition may be in admixture with a carrier or excipient. The carrier or excipient may comprise non-toxic compounds that may be active or inactive and that do not greatly inhibit the nootropic effects of the disclosed composition. The carrier or excipient may also comprise compounds that stabilize the disclosed composition. Furthermore, during the period of time after production of the disclosed composition and before its consumption, the potential of the disclosed composition to enhance some cognitive functions of a user upon consumption by the user may decline with time. As such, the carrier or excipient may slow this decline.

The carrier or excipient may also ease the consumption of the disclosed composition. As non-limiting examples, the carrier or excipient may ease consumption of the disclosed composition by bulking up the composition to increase its volume, by reducing its viscosity for easier ingestion if it is in liquid form, or by increasing the solubility in a liquid for consumption in liquid form, as consumption in a liquid form may be easier for some users than consumption in a solid form.

Furthermore, the carrier or excipient may enhance the visual appeal of the composition by adding color, such as with food coloring. The carrier or excipient may also enhance the taste of the composition, such as by adding natural or artificial flavorings.

The carrier or excipient may comprise, as non-limiting examples, sugar, starch, cellulose, powdered tragacanth, malt, gelatin, talc, cocoa butter, suppository wax, oil, mineral oil, food coloring, natural flavorings, artificial flavorings, citric acid, glycol, polyol, ester, agar, buffering agent, alginic acid, isotonic saline, Ringer's solution, ethyl alcohol, polyester, polycarbonate polyethylene glycol, polyvinylpyrrolidone, water, polyanhydride, antiadherents such as magnesium stearate, or other compound that is non-toxic and that does not greatly inhibit the nootropic effects of the disclosed composition.

The disclosed composition, along with any carrier or excipient that may be added to it, may be orally administered as a liquid, tablet, powder, troche, capsule, elixir, suspension, syrup, wafer, chewing gum, or food.

Preferred amounts of noopept within the disclosed composition are about 1 mg to about 100 mg, about 3 mg to about 50 mg, about 5 mg to about 40 mg, about 10 mg to about 30 mg, or any intervening range therein. In particular preferred embodiments, the disclosed composition comprises about 5 mg, 10 mg, about 20 mg, about 30 mg, or any intervening amount of noopept therein.

Preferred amounts of CBD within the disclosed composition are about 0.5 mg to about 2000 mg, about 1 mg to about 1000 mg, about 2 mg to about 500 mg, about 3 mg to about 300 mg, about 5 mg to about 125 mg, about 10 mg to about 60 mg, about 25 mg to about 50 mg, or any intervening range therein. In particular preferred embodiments, the disclosed composition comprises about 2 mg, about 5 mg, about 10 mg, about 25 mg, about 50 mg, about 100 mg, or any intervening amount of CBD therein.

In one embodiment of the present invention, the composition further comprises a high-concentration choline source. In this embodiment, the high-concentration choline source provides choline to the bloodstream, assisting in preventing the depletion of choline in the brain of the user after consumption of the composition. Not wishing to be bound by any particular theory or model, a possible advantage of this embodiment may be that by providing a sufficient supply of choline for the production of ACh, the high-concentration choline source may further potentiate the nootropic benefits of noopept, such that this particular embodiment may enhance some cognitive functions to a still greater extent than the embodiment not containing a high-concentration choline source. Nevertheless, this possible advantage is not required for this embodiment.

The high-concentration choline source may comprise, for example, at least one of alpha GPC and citicoline. Alpha GPC may be especially effective at providing choline to neurons in the brain because it rapidly transfers choline across the blood-brain barrier. Alpha GPC may be used in the disclosed composition in natural or synthetic forms, as well as from vendors such as, but not limited to, GNC and The Vitamin Shoppe.

Preferred amounts of the high-concentration choline source within the disclosed composition are about 1 mg to about 10,000 mg, about 2 mg to about 5000 mg, about 3 mg to about 2000 mg, about 5 mg to about 1200 mg, about 10 mg to about 600 mg, about 25 to about 250 mg, or any intervening range therein. In particular preferred embodiments, the disclosed composition comprises about 10 mg, about 25 mg, about 50 mg, about 100 mg, about 250 mg, about 600 mg about 1200 mg, or any intervening amount of the high-concentration choline source therein.

In another embodiment, the disclosed composition also comprises caffeine. The caffeine may provide mental stimulation to the user, which may further enhance some cognitive functions, such as mental attention, alertness, reaction time, and mental focus. Users of caffeine, however, are known to develop tolerance to the stimulating effects of caffeine after a period of regular consumption. As such, this particular embodiment may cause users to develop a tolerance to the effects of the caffeine, unlike the previous embodiments, to which users generally do not develop tolerance.

Preferred amounts of the caffeine within the disclosed composition are about 1 mg to about 500 mg, about 5 mg to about 400 mg, about 10 mg to about 250 mg, about 40 mg to about 200 mg, or any intervening range therein. In particular preferred embodiments, the disclosed composition comprises about 20 mg, about 40 mg, about 100 mg, about 200 mg, or any intervening amount of caffeine therein.

Non-limiting examples of methods of administering the disclosed composition to enhance some cognitive functions of the user will now be described. In one embodiment, a fixed quantity of the disclosed composition may be administered orally each day until the user no longer wishes to experience the enhancement of cognitive functions that the disclosed composition may provide. Alternatively, in another embodiment, the consumption of the disclosed composition may be guided by a regimen, wherein the regimen may comprise of at least one of a loading phase near the beginning of the regimen and a period of time during which the user refrains from consumption of the disclosed composition near the end of the regimen, the latter of which will be referred to as the “cleansing phase.”

In a regimen comprising a loading phase, the loading phase may last from about 1 day to about 30 days, from about 2 days to about 20 days, from about 3 days to about 10 days, from about 4 days to about 7 days or some intervening length of time. The preferred length of the loading phase is about 3 days, about 4 days, about 5 days, about 7 days, about 10 days, about 14 days, or about 28 days, or some intervening length of time.

In a regimen comprising a loading phase, the daily dose during the loading phase may be about 10% w/w to about 2000% w/w, about 20% w/w to about 1000% w/w, about 50% w/w to about 500% w/w, about 100% w/w to about 250% w/w larger than the daily dose administered during the remaining part of the regimen, excluding the “zero” daily dose of a possible cleansing phase, or any intervening amount therein. In particular preferred embodiments, the daily dose during the loading phase is about 100% w/w, 250% w/w, or 500% w/w larger than the daily dose during the remaining part of the regimen, or any intervening amount therein.

In a regimen comprising a cleansing phase, the cleansing phase may last from about 1 day to about 1 year, from about 3 days to about 6 months, from about 5 days to about 3 months, from about 7 days to about 28 days, or any intervening range therein. In particular preferred embodiments, the cleansing phase lasts about 3 days, about 5 days, about 7 days, about 14 days, or about 28 days, or any intervening length of time therein.

The aforementioned regimen may last from about 1 day to 1 year, from about 4 days to about 6 months, from about 7 days to about 4 months, from about 14 days to about 2 months, or any intervening range therein, and may be repeated as many times as desired. In particular preferred embodiments, the regimen lasts about 7 days, about 10 days, about 14 days, about 28 days, or any intervening length of time therein.

The disclosed composition has many advantages, some of which, but not all of which, are described here. Not all advantages are required by all embodiments of the composition.

This disclosure has described embodiments as examples, but has not described all possible embodiments of the composition or associated methods. Where a particular feature is disclosed in the context of a particular embodiment, that feature can also be used, to the extent possible, in combination with and/or in the context of other embodiments. Insubstantial modifications of the invention, not presently foreseen, may nonetheless represent equivalents thereto.

What is claimed is:

1. A composition comprising N-Phenylacetyl-L-prolylglycine ethyl ester in combination with cannabidiol, wherein said composition is in admixture with a carrier or excipient comprising a sugar, starch, cellulose, powdered tragacanth, malt, gelatin, talc, cocoa butter, suppository wax, oil, mineral oil, food coloring, natural flavorings, artificial flavorings, citric acid, glycol, polyol, ester, agar, buffering agent, alginic acid, isotonic saline, Ringer's solution, ethyl alcohol, polyester, polycarbonate polyethylene glycol, polyvinylpyrrolidone, water, polyanhydride, or magnesium stearate, wherein said composition is formulated for oral administration.

2. The composition of claim 1, wherein the cannabidiol is isolated from a natural or synthetic source.

3. The composition of claim 1, wherein the formulation comprises a liquid, tablet, powder, troche, capsule, elixir, suspension, syrup, wafer, chewing gum, or food.

4. The composition of claim 1, claim 2, or claim 3 further comprising a compound that comprises choline or is a prodrug for choline, such that introducing the compound into the body of the user results in the concomitant increase of the amount of choline in the bloodstream of the user by an amount greater than or equal to 10% of the mass of the compound.

5. The composition of claim 4 wherein the compound comprises at least one of L-Alpha glycerylphosphorycholine and citicoline.

6. The composition of claim 1 further comprising caffeine.

7. A method for administering a composition comprising N-Phenylacetyl-L-prolylglycine ethyl ester in combination with cannabidiol, wherein said composition is in admixture with a carrier or excipient comprising a sugar, starch, cellulose, powdered tragacanth, malt, gelatin, talc, cocoa butter, suppository wax, oil, mineral oil, food coloring, natural flavorings, artificial flavorings, citric acid, glycol, polyol, ester, agar, buffering agent, alginic acid, isotonic saline, Ringer's solution, ethyl alcohol, polyester, polycarbonate polyethylene glycol, polyvinylpyrrolidone, water, polyanhydride, or magnesium stearate, wherein said composition is formulated for oral administration, and wherein said method comprises a regimen for the daily consumption of the composition.

8. The method of claim 7, wherein the regimen for the daily consumption of the composition comprises a “loading phase,” during which the user consumes an elevated dose of the composition as compared to the dose that the user consumes during the remainder of the regimen.

9. The method of claim 7, wherein the regimen for the daily consumption of the composition comprises a “cleansing phase,” during which the user refrains from consuming the composition, and which is shorter than the regimen.

10. The method of claim 7, wherein the cannabidiol is isolated from a natural or synthetic source.

11. The method of claim 7, wherein the formulation of the composition comprises a liquid, tablet, powder, troche, capsule, elixir, suspension, syrup, wafer, chewing gum, or food.

12. The method of claim 7, wherein the composition further comprises a compound that comprises choline or is a prodrug for choline, such that introducing the compound into the body of the user results in the concomitant increase of the amount of choline in the bloodstream of the user by an amount greater than or equal to 10% of the mass of the compound.

13. The method of claim 12, wherein the compound comprises at least one of L-Alpha glycerylphosphorycholine and citicoline.

14. The method of claim 7, wherein the composition further comprises caffeine.
